(19) United States
(12) Patent Application Publication
(10) Pub. No.: US 2019/0010453 A1
(43) Pub. Date: Jan. 10, 2019

(54) RETINAL STEM CELLS

(71) Applicant: Roberto PINELLI, Paradiso (CH)

(72) Inventor: Roberto PINELLI, Paradiso (CH)

(21) Appl. No.: 16/065,795

(22) PCT Filed: Dec. 20, 2016
(86) PCT No.: PCT/IB2016/057830

§ 371 Date: Jun. 23, 2018

(30) Foreign Application Priority Data
Dec. 23, 2015 (CH) 01907/15

(51) Int. Cl. C12N 5/079; A61K 35/30

(52) U.S. Cl. CPC C12N 5/0621; C12N 2509/00; C12N 2501/10; A61K 35/30

(57) ABSTRACT

The present invention regards stem cells isolated from the ora terminalis and ora serrata regions of the retina, a method for isolating said stem cells, and their use in the treatment of diseases and other pathological conditions that affect the eyes.

### TECHNICAL FIELD

The present invention relates to stem cells isolated from specific regions of the retina, to the method for isolating said stem cells and to their use in the treatment of diseases and other pathological conditions affecting the eyes.

### BACKGROUND ART

Loss of sight can be caused by diseases or damage to the retina or to the eye. For example, the infective process such as the infection of the retina by cytomegalovirus can lead to loss of visual field, reduced visual acuity and blindness. Inflammatory processes such as uveitis also can influence the retina and can lead to a reduction in visual acuity. Cancer of the retina, such as for example retinoblastoma, also worsens vision. Other diseases can occur due to macular degeneration linked to age. Many different genetic diseases, such as retinitis pigmentosa, lead to retinal damage and blindness as well as to other types of retinal degeneration (retinal dystrophies). Physical damage to the retina can also arise from the detachment of the retina, which leads to retinal degeneration and to blindness.

The therapies available for treating the loss of vision caused by retinal damage remain limited.

Said therapies often entail surgical procedures and/or tissue transplant, thus exposing the affected subject to the risk of adverse reactions, which in the case of tissue transplant can comprise the risk of reactions to foreign objects and rejection of the transplant. Furthermore, instead of curing or correcting the underlying disease, many available therapies simply treat or mask the symptoms of these diseases. There remains a need for new therapies for the treatment of diseases and other pathological conditions affecting the eyes, and therapies capable of correcting effectively and permanently these diseases or pathological conditions are particularly necessary. Therapies capable of minimizing the incidence of adverse reactions also are necessary.

The ora terminalis and ora serrata are different parts of the retina, despite being close and connected, see FIG. 1. The ora serrata (“serrata”, from Latin, means “closed”) is the connection between the functional retina and the ora terminalis (“terminalis”, from Latin, means “end part”). The ora terminalis is the most eccentric border of the retina (Holden et al., Retinal Magnification Factor at the Ora Terminalis: A Structural Study of Human and Animal Eyes, Vision Res. 27: 1229-1235, 1987); the ora terminalis is arranged in front of the ora serrata and to the rear of the pars plana, which is part of the uvea. The ora terminalis and the ora serrata are the germinal part of the neural retina in the adult and are the surviving residue of the embryonic neuroepithelial germinal layer that used to cover the entire presumptive neural retina.

The ora terminalis and ora serrata of fish, amphibians and birds are proliferative zones described in various publications. In fish and amphibians, neurogenesis does not cease after the embryonic stage but continues to operate throughout the life of the animal. Stem cells isolated from ora terminalis of fish or amphibians and stem cells isolated from ora serrata of birds have been described, for example by: Amato et al., Retinal stem cells in vertebrates: parallels and divergences, Int. T. Dev. Biol. 48: 993-1001, 2004); Cerveny et al., Continued Growth and Circuit Building in the Anamniote Visual System, Develop. Neurobiol. 72: 328-345, 2012).

The zone at the boundaries of the retinal field adjacent to the ora terminalis has also been widely studied in mammals and in bird species in the context of a study on magnification. The anatomical measurements of the ora serrata in humans and of the ora terminalis in apes, cats, pigeons, cows and pigs were taken as a basis for the calculation of the magnification factors respectively of the ora serrata and ora terminalis. In the eye of humans and apes, the magnification at the extreme peripheral region of the retinal field has been found to be substantially smaller than that at the posterior pole; in the cat, rabbit, rat and mouse there is a lower reduction; in pigeons, tawny owls and starlings, the magnification is very similar at the extreme peripheral region and at the posterior pole (Holden et al., Retinal Magnification Factor at the Ora Terminalis: A Structural Study of Human and Animal Eyes, Vision Res. 27: 1229-1235, 1987).

It is known that retinal stem cells are present in the retina of the adult mammal; specifically, retinal stem cells have been found in the ciliary margin of the retinal epithelium pigmentosum (see U.S. Pat. No. 6,117,675 and Coles et al., PNAS 101(44):15772-15777, 2004) and in the neural retinal layer (see WO 01/58460).

However, the isolation of stem cells from ora terminalis and ora serrata of adult mammals has never been described.

### SUMMARY OF THE INVENTION

Current treatments with retinal stem cells are inadequate for restoring lost vision when the retinal cells are damaged, and therefore the potential clinical applications of retinal stem cells are still very high.

Anatomically, cells that are isolated more deeply in the functional retina have a lower likelihood of being a source of stem cells.

Surprisingly, the Applicant has isolated retinal stem cells from the ora terminalis and ora serrata regions of the retina of adult mammals. The stem cells isolated by the Applicant are capable of producing multipotent cells for the entire life in adult mammals and are a pre-passage to the functional retina.

The retinal stem cell of the ora terminalis (OTRSC) and of the ora serrata (OSRSC) according to the present invention is capable of growing and germinating in neural retinal stem cells, in a manner similar to what occurs in a tree which grows by stratifications of circular rings of cells around the circumference of its trunk. Said OTRSC and OSRSC is capable of withstanding the replacement of retinal cells and regrowth both in healthy human adults and in humans with retinal diseases.

U.S. Pat. No. 6,117,675 and Coles et al., (2004), cited previously, describe retinal stem cells isolated from the region of the ciliary margin of the retina of mammals, i.e., a region that is very distant from the ora terminals and ora serrata; furthermore, the retinal cells described therein are progenitor cells and therefore are unipotent and capable only of producing a progeny which gives rise to a single type of differentiated neural retinal cell.

WO01/58460 cited previously describes retinal stem cells isolated from an anatomically indistinct region of neural retinal tissue of mammals; the isolation of the ora terminalis or ora serrata regions of the retina is not mentioned in this patent application.

On the contrary, the retinal stem cells described in the present invention have been isolated from anatomically distinct regions, i.e., respectively from the ora terminalis and ora serrata regions of the retina of adult mammals, and are multipotent.

The first object of the present invention is therefore an isolated retinal stem cell in which said retinal stem cell is isolated from the ora terminalis and/or ora serrata region of the retina of a postnatal mammal.

The second object of the present invention is a method for isolating a retinal stem cell from a postnatal mammal, in which the method comprises the steps of:

(a) dissociating all or part of the ora terminalis and/or ora serrata from the eye of a subject,
(b) culturing said dissociated ora terminalis and/or ora serrata in a medium that leads to the forming of spheres and/or small groups (clusters) of cells that comprise retinal stem cells,
(c) arranging the resulting spheres and/or clusters of cells in a medium that comprises the enzymes trypsin and hyaluronidase,
(d) blocking the enzymes with one or more inhibitors, and
(e) isolating the resulting retinal stem cells from the resulting spheres and/or clusters of cells.

The third object of the present invention is an isolated retinal stem cell obtained with the method described above.

The fourth object of the present invention is a pharmaceutical composition comprising
an isolated retinal stem cell as defined above or obtained with the method described above, and/or its progeny, and
at least one pharmaceutically acceptable vehicle and/or excipient.

The fifth object of the present invention is the isolated retinal stem cell as defined above or the isolated retinal stem cell obtained with the method described above, and/or its progeny, for use as a medicine.

The sixth object of the present invention is the isolated retinal stem cell as defined above or the isolated retinal stem cell obtained with the method described above, and/or its progeny for use in the treatment of an ophthalmic pathological condition or disease.

The seventh object of the present invention is the use of the isolated retinal stem cell as defined above or of the isolated retinal stem cell obtained with the method described above, and/or of its progeny to produce a medicament for the treatment of an ophthalmic pathological condition or disease.

The eighth object of the present invention is a method for the treatment of an ophthalmic pathological condition or disease in a subject which comprises the step of administering to said subject requiring said treatment an effective quantity of the isolated retinal stem cells as defined above or isolated retinal stem cells obtained by means of the method described above and/or their progeny.

### DEFINITIONS

The retinal stem cells isolated from the ora terminalis are referred to herein as “ora terminalis retinal stem cells” or “OTRSC”.

Said OTRSC are isolated from the eye of a mammal from the period that begins from the first stages of postnatal life and continues through, and sometimes beyond, the eighth decade of life.

The retinal stem cells isolated from the ora serrata are referred to herein as “ora serrata retinal stem cells” or “OSRSC”. Said OSRSC are isolated from the eye of a mammal in the period that begins from the first stages of postnatal life and continues through, and sometimes beyond, the eighth decade of life.

As used herein, the term “isolate” refers to a physical separation or selection of these cells from their native tissues or from their environment. Likewise, the term “isolating” generally references the physical separation or selection of one or more cells from a group of cells (for example a neurosphere), for example according to the characteristics of said cells or to the expression of one or more or cellular or biological markers.

As used herein, the term “mammal” refers to any member of the class of mammals: humans, nonhuman primates such as chimpanzees, and other ape species; farm animals such as bovines, horses, sheep, goats, pigs; pets such as rabbits, dogs and cats; laboratory animals comprising rodents, such as rats, mice and guinea pigs, and the like.

As used herein, the term “undifferentiated” refers to a cell that has not yet differentiated or otherwise developed into a type of specialized cell; stem cells are undifferentiated. Undifferentiated stem cells maintain the ability to differentiate into one or more types of specialized cells (for example cells of the neural retina).

As used herein, the term “multipotent” or “multipotential” refers to cells that are capable of producing a progeny that gives rise to each one of the main types of differentiated cells of the tissue in which they are located. Adult stem cells are multipotent and are capable of producing only functional derivatives. For example, adult retinal stem cells are multipotent and as such are capable of differentiating into one or more specialized neural retinal cells (such as for example amacrine cells, horizontal/off bipolar cells, photoreceptor rods, protein of the external disk of the rod, Muller astrocytes/glia, RPE, pigmented cells, undifferentiated and Mueller neural glia, RPC and amacrine cells, RPC and bipolar rod cells.).

As used herein, the term “unipotent” refers to cells that are capable of producing only one type of cell and are also termed precursor cells.

As used herein, the term “progeny” references the specialized cells that derive from multipotent stem cells.

A therapeutically effective quantity of the isolated retinal stem cells according to the first object of the present invention can be administered to a subject after determining that the subject has a disease or an unwanted condition that can benefit from treatment with said compound. The medical or clinical personnel can make this determination as part of a diagnosis of a disease or pathological condition in a subject. The compound can also be used in the prevention of these conditions, which can be considered as reducing the likelihood that a subject has one or more of the conditions.

As used herein, a “therapeutically effective quantity” refers to a quantity that is sufficient to achieve the intended purpose. The determination of the effective quantities is within the skills of the experts in the field on the basis of the achievement of a desired effect. An effective quantity depends on factors which comprise, without being limited to, the size of a subject and/or the degree to which the disease or unwanted condition affecting the subject has progressed. The effective quantity depends also on the fact that the compound is administered the subject in a single dose or periodically over time. The isolated retinal stem cells according to the first object of the present invention are designed for the treatment of subjects. As used herein, the term “subject” comprises mammals and non-mammals.

Examples of mammals comprise, without being limited to, any member of the class of mammals: humans, nonhuman primates such as chimpanzees and other ape species; farm animals such as bovines, horses, sheep, goats, pigs; pets such as rabbits, dogs and cats; laboratory animals comprising rodents, such as rats, mice and guinea pigs and the like. Examples of non-mammals comprise, without being limited to, birds, fish and the like. Said subject, as understood herein, can be in a period of life from the first stages of postnatal life to over 8 decades of life.

The term “treatment” or “treating” or “treated” as used in the present document comprises the curing or resolution of a pathological disorder in order to obtain a therapeutic benefit, where therapeutic benefit is understood to refer to the eradication or improvement of the underlying disorder to be treated. Furthermore, a therapeutic benefit is reached with the eradication or improvement of one or more of the physiological symptoms associated with the underlying disorder, so that an improvement of the patient is observed despite the fact that the patient may still be subject to underlying disorders.

As used in the present document, “ophthalmic pathological condition or disease” comprises a disease or pathological condition that has an ophthalmic etiology and a disease or pathological condition that has an ophthalmic component that is secondary to a cardiovascular or metabolic disease (such as hypertension, dyslipidemia or diabetes mellitus).

The article “a” and “an”, as used herein in the description and the claims, unless clearly indicated otherwise, must be understood as comprising plural references. The claims or descriptions that comprise “or” between one or more members of a group are considered met if one, more than one, or all the members of the group are present in, used in or otherwise relevant for a given product or process unless indicated otherwise or if this is otherwise evident from the context. The invention comprises embodiments in which exactly one member of the group is present in, used in or otherwise relevant for a given product or method. The invention also comprises embodiments in which more than one, or all the members of the group, are present in, used in, or otherwise relevant for a given product or method. Furthermore, it will be understood that the invention comprises all the variations, combinations and permutations in which one or more limitations, elements, clauses, descriptive terms, etc., in one or more of the listed claims is introduced in another dependent claim of the same claim (or, as relevant, any other claim) unless otherwise indicated or unless it is evident to those who have ordinary experience in the field that a contradiction or inconsistency might derive from it. When elements are represented as lists (in the Markush group or a similar format), it shall be understood that this subgroup of elements is also described, and any element or elements can be removed from the group. It shall be understood also that in general, when the invention or aspects of the invention is/are described as comprising particular elements, characteristics, etc., certain embodiments of the invention or aspects thereof consist, or essentially consist of, said elements, characteristics, etc. For reasons of simplicity, these embodiments have not been indicated specifically in every case. One might also understand that any embodiment or aspect of the invention can be explicitly excluded from the claims, regardless of the fact that the specific exclusion is indicated in the description.

### DESCRIPTION OF THE FIGURE

FIG. 1 is a view of a detail of the sagittal diagram of the eye, which clearly illustrates the respective positions of ora serrata, ora terminalis and retinal ciliary margin.

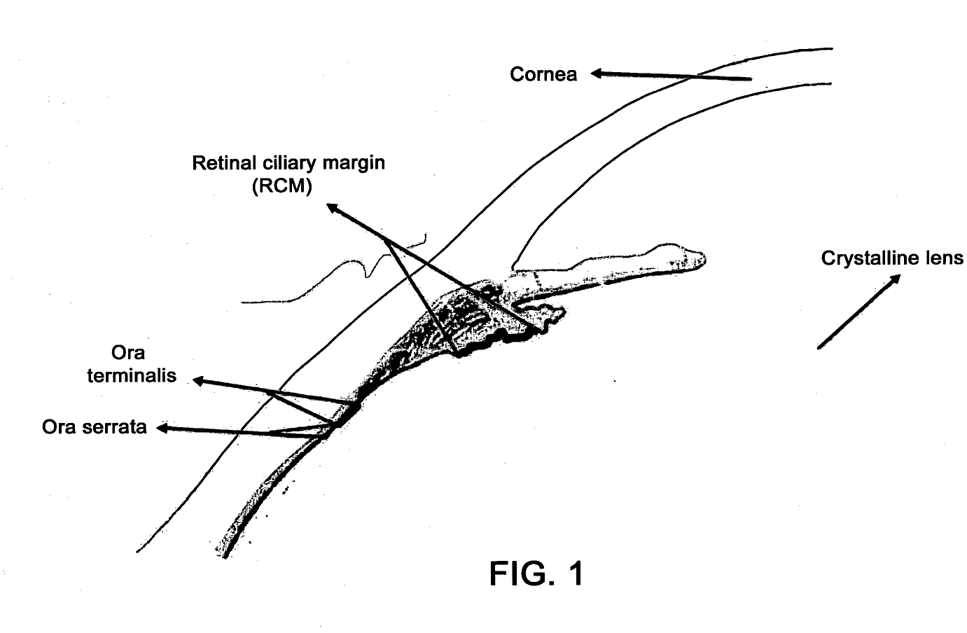

### DETAILED DESCRIPTION OF THE INVENTION

The present invention provides evidence of the fact that the tissues of the eye, and in particular the pigmented and non-pigmented epithelial layers of the region of the ora terminalis and ora serrata of the retina of a postnatal mammal contain retinal stem cells.

The retinal stem cells of the ora terminalis (OTRSC) and the retinal stem cells of the ora serrata (OSRSC) described herein are multipotent and isolated from an anatomically distinct region of the retina and therefore can be distinguished from the retinal stem cells isolated from areas of the ciliary margin (pars plana and pars plicata, parts of the uvea) described in U.S. Pat. No. 6,117,675 and by Coles et al. (2004), which are progenitor cells and therefore are unipotent.

WO01/58460 previously cited describes retinal stem cells isolated from an anatomically indistinct region of the neural retinal tissue of mammals; the isolation from the ora terminalis or ora serrata regions of the retina is not mentioned in this patent application.

The first object of the present invention is an isolated retinal stem cell, wherein said retinal stem cell is isolated from the ora terminalis region and/or from the ora serrata region of the retina of a postnatal mammal.

In an embodiment according to the first object of the present invention, said retinal stem cell is isolated from a region of the retina that comprises said ora terminalis and/or ora serrata region: preferably, said retinal stem cell is isolated from a region of the retina that is constituted by said ora terminalis and/or ora serrata region; more preferably, said retinal stem cell is isolated from a region of the retina that is constituted essentially by said ora terminalis and/or ora serrata region; with maximum preference, said retinal stem cell is isolated from a region of the retina that is constituted exclusively by said ora terminalis and/or ora serrata region and is not isolated from other areas of the retina.

As used herein, the expression “essentially from” indicates that over 75%, preferably more than 80%, more than 85%, more than 90%, more than 95% of the region of the retina from which the retinal stem cell is isolated is constituted by ora terminalis and/or ora serrata.

As used herein, the expression “exclusively from” indicates that over 96%, preferably over 99%, of the region of the retina from which the retinal stem cell is isolated is constituted by ora terminalis and/or ora serrata.

In an embodiment according to the first aspect of the present invention, said OTRSC and/or OSRSC is isolated from the pigmented or non-pigmented epithelial layer; preferably OTRSC and/or OSRSC is isolated from the pigmented layer.

In another embodiment according to the first object of the present invention, said isolated OTRSC and/or said OSRSC is a neural stem cell.

In an embodiment according to the first object of the present invention, said OTRSC and/or OSRSC is isolated during any period of the life of the subject, or in certain cases from a deceased subject (for example said OTRSC and/or OSRSC are isolated within approximately one, two, three, four, six, eight, twelve, twenty-four, thirty-six, forty-eight or seventy-two hours after the death of the subject). Preferably, said subject is a human subject.

Preferably, said OTRSC and/or OSRSC is isolated from a human subject from the period beginning from the early stages of postnatal life and continues through, in some cases beyond, the eighth decade of life.

In an embodiment according to the first object of the present invention, said OTRSC and/or OSRSC is undifferentiated and multipotent; preferably, said OTRSC and/or OSRSC is multipotent and capable of differentiating into all different types of neural retinal cells.

In another embodiment according to the first object of the present invention, said OTRSC and/or OSRSC is not a progenitor cell. In another embodiment according to the first object of the present invention, said OTRSC and/or OSRSC is not unipotent.

The second subject-matter of the present invention is a method for isolating a retinal stem cell from a postnatal mammal, wherein the method comprises the steps of:

(a) dissociating all or part of the ora terminalis and/or ora serrata from the eye of a subject,
(b) culturing said ora terminalis and/or ora serrata that has been dissociated in a medium that leads to the forming of spheres and/or groups (clusters) of cells comprising retinal stem cells,
(c) arranging the resulting spheres and/or the resulting clusters of cells in a medium that comprises the enzymes trypsin and hyaluronidase,
(d) blocking the enzymes with one or more inhibitors, and
(e) isolating the resulting retinal stem cells from the spheres and/or from the resulting clusters of cells.

In an embodiment according to the second object of the present invention, the subject is a human mammal, for example a donor or a cadaver.

In another embodiment according to the second object of the present invention, the medium comprises one or more exogenous growth factors; in another embodiment said medium does not comprise exogenous growth factors.

In an embodiment according to the second object of the present invention, the OTRSC and/or OSRSC are isolated from the corresponding resulting spheres and/or from the clusters of cells with a frequency of approximately 1:500 or at least 1:500 by using routine means such as pipetting.

The third object of the present invention is an isolated retinal stem cell obtained with the method described above.

The fourth object of the present invention is a pharmaceutical composition comprising
an isolated retinal stem cell as defined above or obtained with the method described above, and/or its progeny, and
at least one pharmaceutically acceptable vehicle and/or excipient.

It should be noted that the pharmaceutical composition according to the fourth object of the present invention can be administered to a subject through any suitable administration pathway, comprising one or more among ophthalmic, intrathecal, topical, transdermal, buccal, sublingual, oral or parenteral administration.

In an embodiment according to the fourth object of the present invention, said pharmaceutical composition can be administered by inhaling; in another embodiment said pharmaceutical composition is administered by means of an intravitreal injection.

In an embodiment according to the fourth object of the present invention, the pharmaceutical composition is a solution or a suspension, preferably suitable for ophthalmic administration, more preferably suitable for intravitreal administration; in the case of administration in the vitreous body, this composition can comprise balanced saline solution or a saline solution buffered with Dulbecco phosphate.

Preferably, according to the fourth object of the present invention, the pharmaceutical composition is an aqueous composition comprising the isolated cells of the invention and/or their progeny suspended in a balanced saline solution at a concentration of at least approximately 10,000 cells per 0.5 μl.

Preferably, according to the fourth object of the present invention, the pharmaceutical composition is designed for injection in the vitreous body.

The method for preparing the pharmaceutical composition according to the present invention and the choice of the pharmaceutically acceptable vehicles and/or excipients are described in detail, for example, in L. William, Remington: The Science and Practice of Pharmacy. Twentieth Edition, Mack Publishing Company. Easton, Pa., (2000).

The fifth object of the present invention is the isolated retinal stem cell as defined above or the isolated retinal stem cell obtained with the method described above, and/or its progeny, for use as a medicine.

The sixth object of the present invention is the isolated retinal stem cell as defined above or the isolated retinal stem cell obtained with the method described above, and/or its progeny for use in the treatment of an ophthalmic pathological condition or disease.

The seventh object of the present invention consists in the use of the isolated retinal stem cell as defined above or of the isolated retinal stem cell obtained with the method described above, and/or of its progeny, for the production of a medicament for treating an ophthalmic pathological condition or disease.

The eighth object of the present invention is a method for treating an ophthalmic pathological condition or disease in a subject, comprising the step of administering to said subject requiring said treatment an effective quantity of the isolated retinal stem cells as defined above or isolated retinal stem cells obtained by means of the method described above, and/or their progeny.

In an embodiment according to the sixth, seventh and eighth object of the present invention, the ophthalmic pathological condition or disease to be treated comprises, without being limited thereto, one or more among retinitis pigmentosa, maculopathy, diabetic retinopathy, hypertensive retinopathy and retinal dystrophies.

In an embodiment according to the eighth object of the present invention, the subject is a mammal, preferably human, more preferably a human adolescent.

In an embodiment according to the eighth object of the present invention, the treatment method comprises the step of administering the isolated cell according to the invention, or the isolated retinal stem cell obtained by means of the method described above, and/or its progeny by injection in the vitreous body.

In an embodiment according to the eighth object of the present invention, the treatment method comprises an autologous transplant in a subject of the isolated cell according to the invention, or of the isolated retinal stem cell obtained by means of the method described above, and/or its progeny; in this embodiment, said isolated cell (OTRSC and/or OSRSC) is isolated from the subject, cultured and then transplanted or returned to the same subject, so as to minimize or eliminate the risk of adverse reaction to a foreign body.

As an alternative, in another embodiment according to the eighth object of the present invention, said OTRSC and/or OSRSC is isolated from a donor (such as for example a mammal cadaver) and subsequently transplanted or administered (for example by injection in the vitreous body) to a subject in order to treat an ophthalmic pathological condition or disease.

### EXPERIMENTAL SECTION

### Example 1

Isolation of retinal stem cells from the ora terminalis of a human eye was performed in the present experiment.

The ora terminalis was dissociated and cultured using an assay for forming a clonal sphere, in which the stem cells form clonally derived spheres.

It should be noted that the stem cells are isolated exclusively from the tissue that is anatomically below the sclerocorneal limbus and practically in the ora terminalis. These cells were isolated with a frequency of approximately 1:500.

The biopsy of the tissue was then placed in a tube containing the enzymes trypsin and hyaluronidase and incubated in a water bath at 37° C. for 15 minutes. The enzymes were then blocked by means of inhibitors and single retinal stem cells were subsequently isolated with a mechanical isolation by delicate pipetting. The cells were then counted in a hemocytometric chamber, pelletized by centrifugation and resuspended in an appropriate volume of saline solution buffered with Dulbecco phosphate (DPBS) in order to reach a concentration of approximately 10,000 cells per 0.5 μl.

According to the same procedure described previously, the isolation of the retinal stem cells from the ora serrata of human eyes was performed.

### Example 2

In the present experiment, in vitro proliferation, long-term self-renewal and potential differentiation of isolated ora terminalis retinal stem cells (OTRSC) were evaluated; both the sphere-forming assay and the single-layer assay were used.

The single primary cells obtained from the ora terminalis were plated on plates with 96 wells at a density of one cell per well in order to be able to test whether the isolated OTRSC were capable of proliferating in order to form clonally derived spheres. The OTRSC gave rise to clonal spheres containing both pigmented and non-pigmented cells. Using the single sphere passage assay, the primary spheres were dissociated and replanted, and the individual spheres exhibited a self-renewal capacity, each single sphere giving rise to one or more new spheres in each subsequent passage. The ora terminalis region of the human eye contains retinal stem cells that demonstrate a compliance with the in vitro growth factor that is similar to that of retinal stem cells isolated from rabbit eye ora terminalis.

The clonal spheres were plated in conditions of differentiation and the differentiation potential of their progeny was evaluated. The spheres produced all the different retinal types (such as for example photoreceptor cells), thus demonstrating the multipotentiality of the isolated OTRSC, which by originating from a germinal area of the neural retina are naturally designed to convert into photoreceptors.

By following the same procedure described above, proliferation in vitro, long-term self-renewal and potential for differentiation of isolated retinal stem cells of ora serrata (OSRSC) were studied.

### Example 3

In the present experiment, the in vivo potential of isolated OTRSC and of their progeny was evaluated.

The spherical dissociated human retinal cells containing OTRSC were transplanted into the eye of adult rabbits. In order to visualize the OTRSC and their progeny so as to transplant them into the eye of the host rabbit, a lentiviral construct was prepared which contained an enhanced green fluorescent protein (EGFP). The OTRSC were infected with the lentiviral particles which lead to the development of the green fluorescent spheres. The green fluorescent spheres were then dissociated into individual cells and resuspended in a balanced Hank saline solution at a concentration of 20,000 cells per 1 μl. Subsequently, by means of an intravitreal injection, approximately 10,000 cells per 0.5 μl were transplanted into the vitreous cavity of an eye of an adult rabbit, which had been previously anesthetized topically with proparacaine drops. These OTRSC were injected by using a pipette for buccal administration, modified with barriers, in order to ensure that the OTRSC of the host rabbit did not get contaminated. The eyelids of the host rabbit were then repositioned together and the awoken animals were returned to their cages. Thirty days after transplant, the rabbits were sacrificed and their eyes were enucleated and fixed in 4% PFA (phosphonoformic acid); the eyes were sectioned at 14 μm on a Bright cryostat. The eye sections were then visualized under a fluorescent microscope in order to reveal the presence of EGFP positive cells, i.e., OTRSC and their progeny.

By following the same procedure described above, the in vivo potential of the isolated OSRSCs and of their progeny was evaluated.

### Example 4

The in vitro differentiation potential of OTRSC isolated from pig and rabbit eyes was evaluated in the present experiment.

OTRSC were isolated as described in example 1 and then their differentiation potential was evaluated as described in example 2.

A percentage of the cells that migrated from the OTRSC spheres remained undifferentiated even after three weeks, but most of the differentiated cells were photoreceptor cells.

The above-cited experiments demonstrate that ora terminalis and ora serrata of mammals are a fertile source of bona fide retinal stem cells characterized by proliferation in vitro, long-term self-renewal and differentiation potential.

The preceding experiments demonstrate furthermore that OTRSC, OSRSC and the respective progeny are capable of surviving in vitro, migrating, integrating and differentiating into cells of the neural retina, and in particular into photoreceptors (i.e., rod cells and cone cells), thus supporting the usefulness of OTRSC and/or OSRSC for the treatment of ophthalmic diseases or pathological conditions.

Furthermore, the experiments described in example 2 demonstrates that in the sphere forming assay each sphere derives from a single cell, suggesting that the rare pigmented cells in the ciliary margin are undifferentiated stem cells. Most of the pigmented cells in the spheres were progenitors of pigmented retinal epithelium.

The human neural retina has shown that it contains retinal progenitor cells that are similar to the retinal progenitor cells isolated from rabbit eye, ensuring support to the idea that the adult mammal eye can contain previously derived retinal stem cells.

1. An isolated retinal stem cell, wherein said retinal stem cell is isolated from the ora terminalis region of the retina of a postnatal mammal, from the ora serrata region of the retina of a postnatal mammal, or a combination thereof.

2. (canceled)

3. The isolated retinal stem cell according to claim 1, wherein said isolated retinal stem cell is undifferentiated and multipotent.

4. A method for isolating a retinal stem cell from a postnatal mammal, comprising:
creating an isolate by dissociating all or part of the ora terminal, the ora serrata or a combination of the ora terminalis and the ora serrata from the eye of a subject;
culturing all or a portion of said isolate in a medium that engenders the formation of spheres, small groups, or clusters of cells that comprise retinal stem cells;
arranging the resulting spheres, small groups or clusters of cells in a medium that comprises the enzymes trypsin and hyaluronidase;
blocking the enzymes with one or more inhibitors; and
isolating the resulting retinal stem cells from the resulting spheres, small groups or clusters of cells.

5. The isolated retinal stem cell according to claim 1, obtained by a process comprising the steps of:
creating an isolate by dissociating all or part of the ora terminalis, of the ora serrata or a combination of the ora terminalis and the ora serrata from the eye of a subject;
culturing all or a portion of said isolate in a medium that engenders the formation of spheres, small groups, or clusters of cells that comprise retinal stem cells;
arranging the resulting spheres, small groups or clusters of cells in a medium that comprises the enzymes trypsin and hyaluronidase;
blocking the enzymes with one or more inhibitors; and
isolating the resulting retinal stem cells from the resulting spheres, small groups or clusters of cells.

6. (canceled)

7. (canceled)

8. The isolated retinal stem cell r of claim 1, wherein said isolated retinal stem cell is adapted for use as medicine.

9. The isolated retinal stem cell of claim 1, wherein said isolated retinal stem cell is adapted for use in the treatment of an ophthalmic pathological condition or disease.

10. The isolated retinal stem cell of claim 9, wherein the ophthalmic pathological condition or disease to be treated is retinitis pigmentosa, maculopathy, diabetic retinopathy, hypertensive retinopathy, retinal dystrophy or a combination thereof.

11. The isolated retinal stem cell according to claim 3, wherein said stem cell is multipotent and capable of differentiating into all the different types of neural retinal cell.

12. The isolated retinal stem cell according to claim 8, wherein said isolated retinal stem cell is adapted to be carried by a pharmaceutical delivery agent to a targeted anatomy.

13. The isolated retinal stem cell according to claim 12, wherein said stem cell and said pharmaceutical delivery agent are configured as an effective dose to be delivered by an ophthalmic, intrathecal, topical, transdermal, buccal, sublingual, oral or parenteral administration pathway.

14. The isolated retinal stem cell according to claim 8, wherein said isolated retinal stem cell is adapted to be carried by a pharmaceutical delivery agent to treat an ophthalmic condition or disease.

15. The isolated retinal stem cell according to claim 14, wherein said ophthalmic condition or disease is retinitis pigmentosa, maculopathy, diabetic retinopathy, hypertensive retinopathy, retinal dystrophy or a combination thereof.

16. The isolated retinal stem cell according to claim 8, wherein said isolated retinal stem cell is adapted for injection into the vitreous body.

17. The isolated retinal stem cell according to claim 8, wherein said isolated retinal stem cell is adapted to be carried by a pharmaceutical delivery agent and injected into the vitreous body.

18. The isolated retinal stem cell according to claim 1, wherein said postnatal mammal is a human.

19. The method of claim 4, wherein said medium comprises one or more exogenous growth factors.

20. The method of claim 4, wherein said retinal stem cells are isolated from the spheres, small groups or clusters of cells with a frequency of about 1:500.

21. A method for the treatment of an ophthalmic pathological condition or disease in a subject, comprising the step of administering to said subject in need of such treatment an effective quantity of the isolated retinal stem cell according to claim 1 and/or its progeny, wherein said ophthalmic pathological condition or disease to be treated is at least one of: retinitis pigmentosa, maculopathy, diabetic retinopathy, hypertensive retinopathy and retinal dystrophy.
